(19) United States
(12) Patent Application Publication
(10) Pub. No.: US 2025/0297201 A1
(43) Pub. Date: Sep. 25, 2025

(54) Non-Invasive Cell Culture Well for Monitoring and Recording Uterine Muscle Contractions

(71) Applicant: The Board of Regents of the University of Texas System, Austin, TX (US)

(72) Inventors: Ananth Kumar Kammala, Austin, TX (US); Ramkumar Menon, Austin, TX (US); Ourlad Alzeus G. Tantengco, Austin, TX (US); Rahul Cherukuri, Austin, TX (US); Han Arum, Austin, TX (US)

(21) Appl. No.: 19/085,766

(22) Filed: Mar. 20, 2025

Related U.S. Application Data
(60) Provisional application No. 63/568,215, filed on Mar. 21, 2024.

(51) Int. Cl. C12M 3/00; B33Y 80/00; C12M 1/12; C12M 1/32; C12M 1/34; C12M 1/36; C12M 1/42

(52) U.S. Cl. CPC C12M 21/08; B33Y 80/00; C12M 23/12; C12M 25/06; C12M 35/00; C12M 41/36; C12M 41/48

(57) ABSTRACT

Various examples are provided related to apparatus that can be utilize for non-invasive monitoring of tissues and other cultures. In one example, an apparatus includes a cell culture well including a first cylindrical member defining a first inner space, a second cylindrical member defining a second inner space, and a channel extending between the first and second inner spaces. The cell culture well includes a column extending upward within the first inner space and an opening provided at a bottom of the second inner space. A sensor can extend through the opening into the second inner space. The sensor can be a columnar sensor that extends upward within the second inner space. A tissue sample can be provided within the cell culture well, where the tissue sample extends between the column and the sensor, and tissue sample activity can be monitored in real-time via the sensor.

### CROSS REFERENCE TO RELATED APPLICATIONS

This application claims priority to, and the benefit of, U.S. provisional application entitled “Experimental Apparatus” having Ser. No. 63/586,215, filed Mar. 21, 2024, which is hereby incorporated by reference in its entirety.

### BACKGROUND

Monitoring uterine muscle contractions is crucial for understanding labor mechanisms, detecting abnormalities, and developing effective tocolytic drugs. However, current methods for observing and recording uterine contractions are often invasive and unsuitable for preclinical drug trials. Accordingly, there is a need for apparatuses and methods for monitoring and recording such contractions.

### SUMMARY

Aspects of the present disclosure are related to apparatus that can be utilize for non-invasive monitoring of tissues and other cultures. In one aspect, among others, an apparatus comprises a cell culture well comprising a first cylindrical member defining a first inner space, a second cylindrical member defining a second inner space, and a channel extending between the first and second inner spaces, the cell culture well further comprising a column extending upward within the first inner space of the first cylindrical member and an opening provided at a bottom of the second inner space of the second cylindrical member.

In one or more aspects, the first and second cylindrical members and the column can extend upwardly from a top surface of a base plate. The channel can be defined by opposed parallel walls that also extend upwardly from the top surface of the base plate. The first and second cylindrical members, the column, the parallel walls, and the base plate can be unitarily constructed of a single material. The single material can be a biocompatible polymer. The cell culture well can be fabricated using a three-dimensional printing process. The channel extending between the first and second inner spaces can comprise a divider wall extending between the opposed parallel walls, the divider wall comprising one or more opening extending through the divider wall adjacent to the base plate.

In various aspects, the apparatus can comprise a columnar sensor that extends through the opening provided at the bottom of the second inner space and upward within the second inner space of the second cylindrical member. The apparatus can comprise an isometric transducer electrically connected to the columnar sensor. The apparatus can comprise a bridge amplifier electrically connected to the isometric transducer. The apparatus can comprise a computer electrically connected to the bridge amplifier, the computer comprising a software application configured to analyze and present data sensed by the sensor to a user.

In another aspect, a method comprises providing an apparatus comprising: a cell culture well comprising a first cylindrical member defining a first inner space, a second cylindrical member defining a second inner space, and a channel extending between the first and second inner spaces; a column extending upward within the first inner space of the first cylindrical member; and a sensor extending through an opening provided at a bottom of the second inner space of the second cylindrical member; providing a tissue sample within the cell culture well, where the tissue sample extends between the column and the sensor; and monitoring tissue sample activity in real-time via the sensor.

In one or more aspects, the tissue sample can be disposed on a top surface of a base plate supporting the first and second cylindrical members and the column. The opening can extend through the based plate and the sensor can be a columnar sensor that extends through the opening. An isometric transducer can be electrically connected to the columnar sensor. In various aspects, the tissue sample can comprise muscle tissue. The tissue sample activity can comprise muscle contractions. The tissue sample activity can be in response to a stimulus. The tissue sample activity can be in response to application of a drug. The method can comprise recording output signals from the sensor. The method can comprise amplifying and conditioning the output signals prior to recording.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of an experimental apparatus that can be used to conduct experiments on a tissue sample, in accordance with various embodiments of the present disclosure.

FIG. 2 illustrates an example of a cell culture well of the experimental apparatus of FIG. 1, in accordance with various embodiments of the present disclosure.

FIG. 3 illustrates an example of a use of the cell culture well of FIG. 2, in accordance with various embodiments of the present disclosure.

FIG. 4 includes images of myometrial cells in GeIMA and Collagen-1, in accordance with various embodiments of the present disclosure.

FIG. 5 illustrates an example of cyclical contraction and relaxation of a myometrial cell culture sensed using an isometric transducer, in accordance with various embodiments of the present disclosure.

FIG. 6 illustrates an example of a cell culture well comprising a divider wall, in accordance with various embodiments of the present disclosure.

### DETAILED DESCRIPTION

Disclosed herein are various examples related to apparatus that can be utilize for non-invasive monitoring of tissues and other cultures. Reference will now be made in detail to the description of the embodiments as illustrated in the drawings, wherein like reference numbers indicate like parts throughout the several views.

As described above, there is a need for apparatuses and methods for monitoring and recording uterine contractions. Disclosed herein are examples of non-invasive and functional apparatuses and methods suitable for that purpose. In one embodiment, an experimental apparatus comprises one or more cell culture wells that facilitate the growth of uterine muscle tissue in vitro and the application of pharmacologic agents to the tissue for the purpose of monitoring and recording responsive uterine muscle contractions. In some embodiments, each culture well comprises two opposed cylindrical members that extend upward from a base plate. Extending between the cylindrical members are opposed walls that also extend upward from the base plate and together define a channel that connects inner spaces defined by the cylindrical members. With such a construction, a dumbbell-shaped well is formed within which uterine muscle tissue can be cultivated and experimented upon. Muscle contractions can be responsive to a range of stimulations (e.g., electrical, chemical, etc.).

Extending upward from the base within one of the cylindrical members is a column. Formed through the base within a space defined by the other cylindrical member is an opening through which a columnar sensor can be passed into the space. Once the uterine muscle tissue has been cultivated within the well and surrounds both the cylindrical column of the first cylindrical member and the columnar sensor extending into the second cylindrical member, one or more agents can be supplied within the well and any contractions can be detected by the sensor and recorded for purposes of evaluation.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

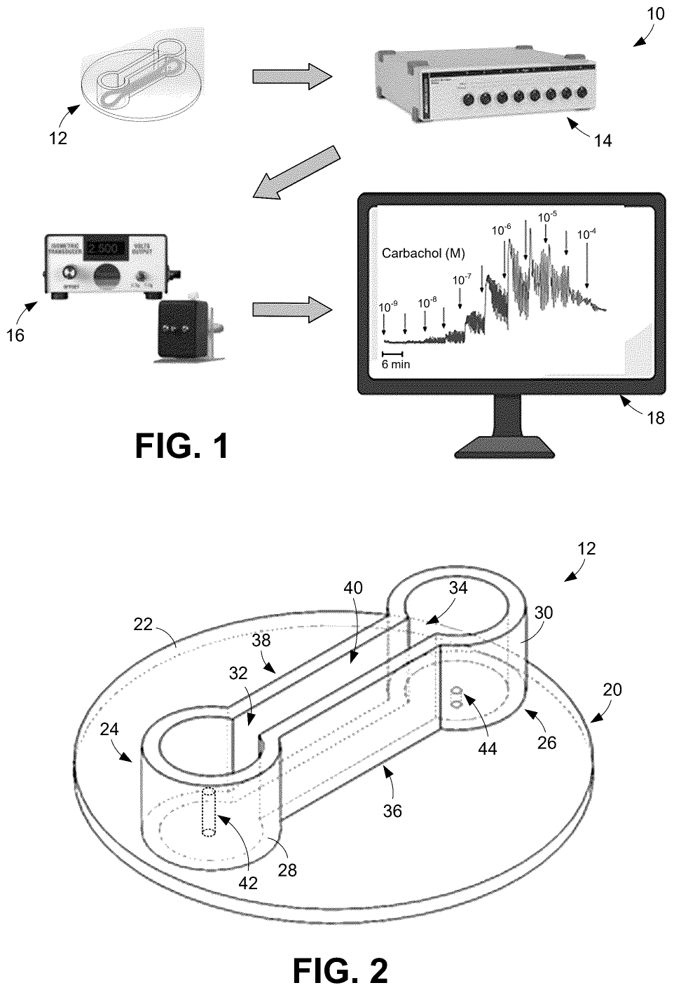

FIG. 1 illustrates an example experimental apparatus 10 that can be used to conduct experiments on living tissue, such as uterine muscle tissue. As shown in the figure, the apparatus 10 generally includes a cell culture well 12, a transducer 14, an amplifier 16, and a computer 18, which is represented by a computer display. As suggested by the arrows present between the above-identified components, signals (i.e., data) sensed from within the well 12 are transmitted to the transducer 14, which then transmits the signals to the amplifier 16 for amplification, and the amplified signals are then transmitted to the computer 18 for recordation and display. Other circuit configurations can also be utilized for the capture, recording and display of data from the cell culture well 12.

FIG. 2 shows the cell culture well 12 in greater detail. As depicted in that figure, the well 12 comprises a base plate 20, which, in the example of FIG. 2, comprises a thin, planar, and circular element. Extending upward from a top surface 22 of the base plate 20 are a first cylindrical member 24 and a second cylindrical member 26. As shown in FIG. 2, each cylindrical member 24, 26 is spaced from the other member on the base plate 20, and each member is formed by a single continuous wall 28, 30 that terminates at opposed ends without forming a complete cylinder, thereby leaving narrow gaps 32, 34 that face each other. Also extending upward from the top surface 22 of the base plate 20 are first and second planar walls 36 and 38 that each extend between an end of the wall 28 forming the first cylindrical member 24 and an end of the wall 30 forming the second cylindrical member 26. With such a configuration, the planar walls 36, 38 define a channel 40 that extends between and connects the inner spaces defined by the cylindrical members 24, 26. As is further shown in FIG. 2, each of the walls 28, 30, 36, and 38 can have the same or similar height as well as the same or similar thickness.

With further reference to FIG. 2, extending upward from the top surface 22 of the base plate 20 within the inner space defined by the first cylindrical member 24 is a column 42. In the example of FIG. 2, the column 42 is concentric with the cylindrical member 24 but has a much smaller diameter than that of the cylindrical member. As described below, tissue grown within the well 12, such as uterine muscle tissue, can wrap around the column 42 so that tension may be applied by or to the tissue. Formed within the inner space defined by the second cylindrical member 26 is a small opening 44 extending through the base plate 20 that, like the column 42, is concentric with its cylindrical member. As described below, a columnar sensor, such as an electrode of the transducer 14, can be passed through the opening 44 and can, like the column 42, provide a structure that the cultured tissue can surround.

Although alternative constructions are possible, in some embodiments the cell culture well 12 is unitarily formed from a single piece of biocompatible material, such as a biocompatible polymer made from, e.g., a mixture of methacrylic esters and photoinitiators produced by Formlabs of Massachusetts, USA, under the name “BioMed Clear Resin.” Although the well 12 can be fabricated using any one of a variety of techniques, in some embodiments the well 12 can be fabricated using a three-dimensional (3D) printing process. It is further noted that, while a single, independent well 12 is illustrated and has been described, it is contemplated that multiple such wells 12 can be integrated together to provide a multi-well device. For example, multi-well plates comprising many (e.g., 96) such wells 12 can be produced to enable multiple experiments to be conducted using a single apparatus.

Irrespective of its particular construction, the cell culture well 12 provides a scaffold that ensures the proper alignment and spatial organization of cultured cells, thereby enabling researchers to study cell behavior and interactions in a biomimetic context. The well 12 provides a safe environment for cells to proliferate and form tissues, which enables a variety of investigations, including tissue engineering, drug screening, and other cellular studies. In the context of the study of uterine contractions, 3D structures composed of human myometrial cells, the smooth muscle cells in the uterine wall, or human cervical stromal cells can be cultivated using bioprinting techniques. Such techniques enable the creation tissue-like structures that mimic the natural tissues within the uterus and the cervix. 3D bioprinted myometrial cells can serve as a model for studying uterine contractions and related physiological processes, while 3D bioprinted cervical stromal cells can serve as a model for studying cervical remodeling processes, including contraction and dilation. Those physiological processes are essential in studying the biology of pregnancy and parturition.

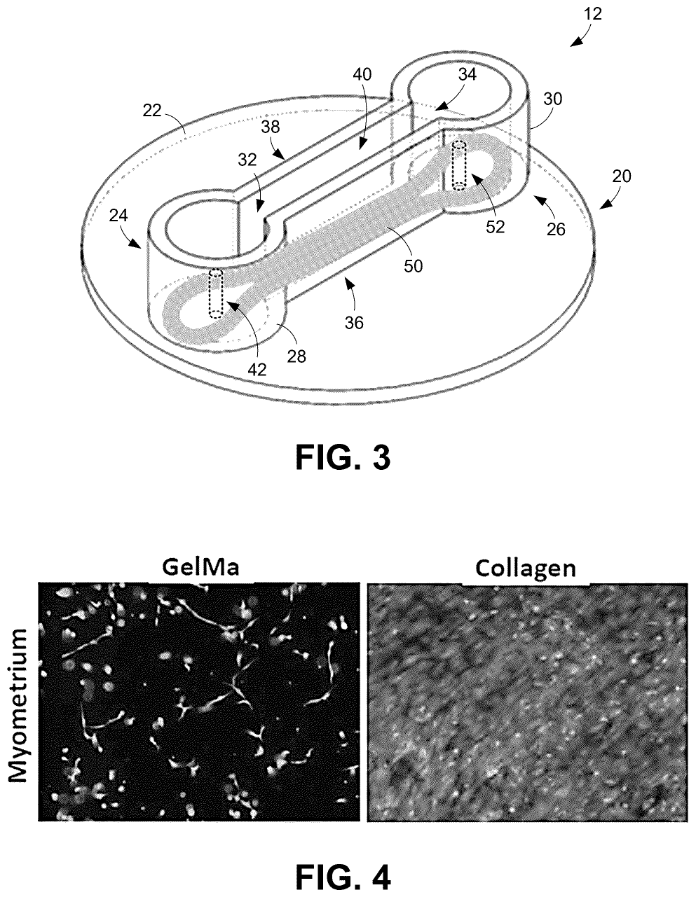

FIG. 3 illustrates an example of use of the cell culture well 12 in which bioprinted tissue 50 has been deposited within the well. As shown in the figure, the tissue 50 is wrapped around the column 42 within the first cylindrical member 24, extends through the channel 40 formed by the walls 36, 38, and is wrapped around a sensor electrode 52 that extends through the opening 44 provided through the base plate 20 within the second cylindrical member 26. The open architecture of the cylindrical members 24, 26 and the channel 40 formed by the walls 36, 38 allows for imaging of the tissue during testing using a wide range of imaging devices.

With reference back to FIG. 1, the transducer 14 can, in some embodiments, comprise an isometric transducer that measures changes in muscle tension or force. In the uterine contraction context, the transducer 14 can be used to detect and quantify the mechanical force generated by 3D bioprinted myometrial cells during contraction. In such a case, the transducer 14 converts the mechanical forces into electrical signals that can be further processed and recorded.

With further reference to FIG. 1, the amplifier 16 can comprise a bridge amplifier that amplifies and conditions the electrical signals output from the transducer 14. In some embodiments, the amplifier 16 can be specifically designed to work with strain gauge-based transducers, such as isometric transducers. By amplifying the signals, the amplifier 16 enables small changes in electrical resistance detected by the transducer 14 to be more easily identifiable and, therefore, facilitates analysis of the signals and the changes within them that are indicative of contraction.

The computer 18 can comprise software that enables the aforementioned analysis of the signals. In some embodiments, existing software applications, such as LabChart™, can be used for this purpose. LabChart™ enables the electrical signals output from the transducer 14 to be visualized, analyzed, and digitally stored. Moreover, LabChart™ provides a user-friendly interface for data recording, real-time visualization, and advanced analysis of uterine contractions.

Through the combination of the various components described above, the experimental apparatus enables researchers to conduct in-depth analysis of uterine contractions and related physiological phenomena. It is noted that, while those components are identified as independent components, in other embodiments, some or all of the components can be integrated into a single system or device, if desired. Regardless, the integration of the disclosed cell culture well in electrophysiology experiments offers additional benefits through multiple downstream analyses. Following electrophysiology experiments, researchers can harvest the 3D cell cultures from the well, enabling further investigations. One advantage of this approach is the ability to subject the harvested cell cultures to various assays, including immunofluorescence staining, Western blot analysis, and omics studies, such as transcriptomics, proteomics, or metabolomics. These molecular assays complement the functional results obtained from the electrophysiology experiments, offering a more comprehensive and integrated understanding of the cellular behavior. The combination of electrophysiology data with molecular insights obtained through downstream analyses enhances the overall research outcomes and contributes to a more comprehensive and nuanced understanding of the investigated biological processes.

One purpose of developing a cell culture well of the type disclosed herein is to monitor smooth muscle contractions with an isometric transducer to create a sophisticated and physiologically relevant model that accurately replicates the architecture and contractile properties of native myometrium. Unlike conventional two-dimensional cell cultures or tissue models, the disclosed well enables a biomimetic approach, providing a more representative environment for studying uterine and cervical smooth muscle behavior during pregnancy and labor.

It is noted that there are many differences between the disclosed cell culture well and existing alternative solutions, such as:
Customizability: The 3D bioprinted device allows researchers to design and tailor its structure, ensuring it closely resembles the specific tissue of interest.
Precision: With precise control over tissue composition, thickness, and arrangement, the device enhances the accuracy and relevance of experimental data.
Real-Time Monitoring: The integration of a forced isometric transducer enables continuous and dynamic monitoring of muscle contractions, providing real-time feedback on myometrial tissue behavior.
Drug Testing and Treatment Evaluation: The 3D bioprinted device offers a more physiological context for drug testing, allowing researchers to assess treatment efficacy in a sophisticated and relevant model. This device can be used to develop new drugs that can be used as cervical ripening agents that can facilitate safe labor and delivery, uterotonics to induce labor and prevent postpartum hemorrhage, and tocolytics that can suppress uterine contractions and delay labor and delivery.
Advancing Pregnancy Research: Studying cervical and uterine biology during pregnancy has indeed posed challenges, primarily due to the difficulty of obtaining tissue samples from pregnant patients. However, this innovative device offers a promising solution by providing a platform to model human uterine and cervical tissues with their contractile functions. By accurately replicating native uterine and cervical smooth muscle tissue and providing controlled experimental conditions, this device represents a significant advancement in pregnancy-related research. Moreover, this technology opens up possibilities for exploring potential therapeutic interventions for pregnancy-related conditions. By testing various treatments and interventions in a realistic model, researchers can identify promising approaches to address pregnancy-related issues, such as preterm labor or cervical ripening.

Moreover, the disclosed cell culture well's ability to accurately replicate native tissue and offer controlled experimental conditions sets it apart for drug testing and treatment evaluation. The well provides a more physiological context for drug studies, potentially leading to more reliable assessments of treatment efficacy. Ultimately, the advanced capabilities of the disclosed well contributes to advancing pregnancy-related research, providing a deeper understanding of uterine and cervical smooth muscle function, and exploring potential therapeutic interventions for pregnancy-related conditions.

Experiments were performed using an experimental apparatus similar to that illustrated in FIG. 1. Myometrial cells were 3D printed using a combination of GeIMA (gelatin methacryloyl) and collagen and uterine muscle contractions were recorded in real time. Cell viability within the different biocompatible materials (GeIMA and collagen) was assessed using live-dead staining. In vitro, experiments were conducted using the disclosed cell culture well to evaluate its functionality and performance by stimulating the cells with acetylcholine at a concentration of 1 μM.

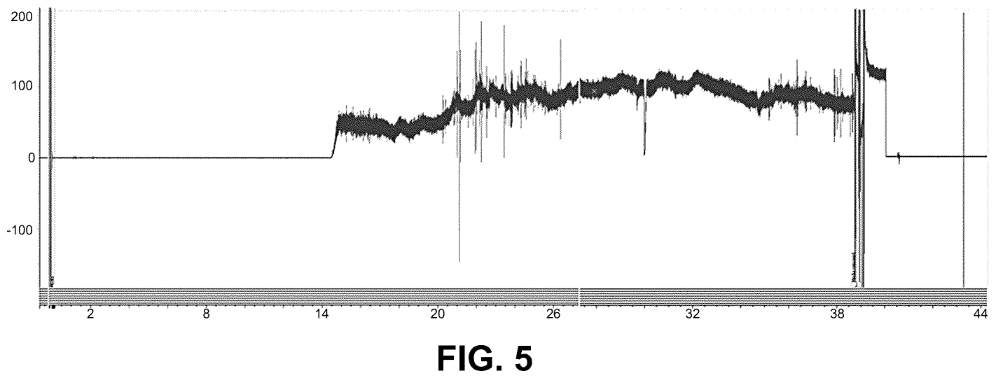

The cell culture well provided a suitable platform for culturing uterine muscle cells and mimicking their physiological environment. Incorporating GeIMA and collagen in the cell media mixture maintained cell viability within the well. Myometrial cells were viable (green) in biocompatible materials like GeIMA and Collagen-1, as shown in FIG. 4. The cyclical contraction and relaxation of the 3D myometrial cell culture was sensed and recorded using an isometric transducer and LabChart™ (FIG. 5). The well successfully replicated the architectural and contractile properties of the native myometrial tissue, providing a conducive environment for culturing uterine muscle cells. These results were obtained through the methods employed to conduct the experiments, including acetylcholine stimulation.

The experiments revealed that the disclosed apparatus presents a promising vehicle for studying uterine physiology and conducting preclinical trials for various childbirth-related interventions, such as uterotonics, tocolytics, and treatments for postpartum bleeding. The apparatus enables real-time monitoring of drug responses to uterine contractions, enabling researchers to study drug efficacy and safety more effectively. Further research and validation will contribute to obstetric advancements, benefitting maternal and fetal healthcare and the development of new therapeutics.

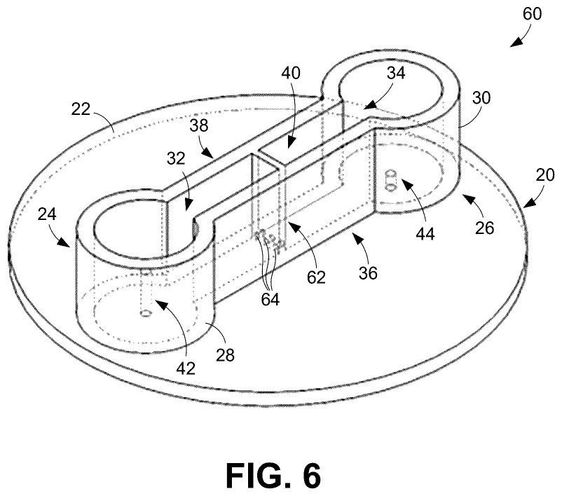

FIG. 6 illustrates an alternative cell culture well 60. The well 60 is similar in construction to that of the well 12 shown in FIG. 2 and, therefore, comprises many of the same elements as that well, which are identified using the same reference numerals as those used in FIG. 2. In addition, however, the well 60 incudes a transverse divider wall 62 that divides the channel 40 generally in half. Provided at the bottom of the wall 62 adjacent the top surface 22 of the base plate 20 are one or more openings 64 (three such openings are illustrated in the example of FIG. 6) that extend through the wall 62 to enable liquid and solids (e.g., cells) to pass from one side of the channel 40 to the other. The divider wall 62 can be used to simultaneously co-culture two different cell types in the well 60 on opposite sides of the wall. The openings 64 enable the different cells to communicate with each other during the culture. In addition or in the alternative, the divider wall 62 can be used to study the effect and propagation of a drug that is introduced on one side of the wall on cells contained on the other side of the wall.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. An apparatus, comprising:
a cell culture well comprising a first cylindrical member defining a first inner space, a second cylindrical member defining a second inner space, and a channel extending between the first and second inner spaces, the cell culture well further comprising a column extending upward within the first inner space of the first cylindrical member and an opening provided at a bottom of the second inner space of the second cylindrical member.

2. The apparatus of claim 1, wherein the first and second cylindrical members and the column extend upwardly from a top surface of a base plate.

3. The apparatus of claim 2, wherein the channel is defined by opposed parallel walls that also extend upwardly from the top surface of the base plate.

4. The apparatus of claim 3, wherein the first and second cylindrical members, the column, the parallel walls, and the base plate are unitarily constructed of a single material.

5. The apparatus of claim 4, wherein the single material is a biocompatible polymer.

6. The apparatus of claim 5, wherein the cell culture well is fabricated using a three-dimensional printing process.

7. The apparatus of claim 3, wherein the channel extending between the first and second inner spaces comprises a divider wall extending between the opposed parallel walls, the divider wall comprising one or more opening extending through the divider wall adjacent to the base plate.

8. The apparatus of claim 1, further comprising a columnar sensor that extends through the opening provided at the bottom of the second inner space and upward within the second inner space of the second cylindrical member.

9. The apparatus of claim 8, further comprising an isometric transducer electrically connected to the columnar sensor.

10. The apparatus of claim 9, further comprising a bridge amplifier electrically connected to the isometric transducer.

11. The apparatus of claim 10, further comprising a computer electrically connected to the bridge amplifier, the computer comprising a software application configured to analyze and present data sensed by the sensor to a user.

12. A method, comprising:
providing an apparatus comprising:
a cell culture well comprising a first cylindrical member defining a first inner space, a second cylindrical member defining a second inner space, and a channel extending between the first and second inner spaces;
a column extending upward within the first inner space of the first cylindrical member; and
a sensor extending through an opening provided at a bottom of the second inner space of the second cylindrical member;
providing a tissue sample within the cell culture well, where the tissue sample extends between the column and the sensor; and
monitoring tissue sample activity in real-time via the sensor.

13. The method of claim 11, wherein the tissue sample is disposed on a top surface of a base plate supporting the first and second cylindrical members and the column.

14. The method of claim 13, wherein the opening extends through the based plate and the sensor is a columnar sensor that extends through the opening.

15. The method of claim 14, wherein an isometric transducer is electrically connected to the columnar sensor.

16. The method of claim 11, wherein the tissue sample comprises muscle tissue.

17. The method of claim 16, wherein the tissue sample activity comprises muscle contractions.

18. The method of claim 11, wherein the tissue sample activity is in response to a stimulus.

19. The method of claim 18, wherein the tissue sample activity is in response to application of a drug.

20. The method of claim 11, comprising recording output signals from the sensor.
